(19) United States
(12) Patent Application Publication
(10) Pub. No.: US 2001/0049363 A1
(43) Pub. Date: Dec. 6, 2001

(54) Oral composition containing NSAIDs and essential oils

(75) Inventors: Michael Rubin, Boonton, NJ (US); Pauline Pan, Morris Plains, NJ (US); Frank Volpe, Kinnelon, NJ (US)

Correspondence Address: Evan J. Federman, Legal Division, Warner-Lambert Company, 201 Tabor Road, Morris Plains, NJ, 07950, US

(73) Assignee: Warner-Lambert Company, Morris Plains, NJ (US)

(21) Appl. No.: 09/761,026

(22) Filed: Jan. 16, 2001

Related U.S. Application Data
(60) Provisional application No. 60/204,375, filed on May 16, 2000.
(60) Provisional application No. 60/084,884, filed on May 8, 1998.
(60) Provisional application No. 60/130,356, filed on Apr. 21, 1999.

(51) Int. Cl. A61K 31/60; A61K 31/045

(52) U.S. Cl. 514/164; 514/729

(57) ABSTRACT

Oral compositions for treating and/or preventing gingivitis are provided. The compositions include at least one NSAID, thymol, methyl salicylate, menthol and eucalyptol. The NSAID, thymol, methyl salicylate, menthol and eucalyptol are present in the composition in synergistically effective amounts. The compositions can be provided in the form of, e.g., mouthwashes or toothpastes, and are not only effective against gingivitis, but can also prevent or treat halitosis and other detrimental conditions of the oral cavity.

### FIELD OF THE INVENTION

This invention relates to oral compositions for treating and/or preventing diseases of the mouth, and more particularly to oral compositions for treating and/or preventing gingivitis.

### BACKGROUND OF THE INVENTION

Gingivitis is a disease characterized by inflammation of the gingiva or gums. It is generally accepted that this inflammation is typically caused by an overabundance of bacterial plaque about the base of the teeth. Thus, a good deal of research has focused on preventing or treating gingivitis by minimizing the amount of bacterial plaque on the teeth and countering the inflammatory response of the gingiva.

The amount of bacterial plaque on the teeth can be controlled by good hygiene, including mechanical removal by frequent brushing, flossing and the like. As an adjunct to the traditional mechanical methods for limiting the amount of bacterial plaque on the teeth, chemical methods have been developed that typically function by killing the bacteria responsible for forming plaque on the teeth.

For example, U.S. Pat. No. 5,472,684 discloses oral compositions purportedly effective in countering plaque and gingivitis. The compositions comprise thymol and eugenol as antimicrobial agents that kill plaque-forming bacteria. U.S. Pat. No. 5,405,604 discloses a concentrated mouthrinse comprising a cationic antimicrobial agent, such as a quaternary ammonium compound, which purportedly reduces the amount of undesirable bacteria in the oral cavity.

LISTERINE® brand mouthwash is, perhaps, the most well-known example of an antiseptic oral composition that has proven effective in killing microbes in the oral cavity that are responsible for plaque, gingivitis and bad breath. LISTERINE® brand mouthwash is believed to achieve its antimicrobial effect through a combination of essential oils that act as antimicrobial agents. These essential oils include precisely balanced amounts of thymol, methyl salicylate, menthol and eucalyptol.

As mentioned earlier, gingivitis can also be prevented and treated with anti-inflammatory agents.

For example, U.S. Pat. No. 4,933,172 discloses the use of meclofenamic acid, a nonsteroidal anti-inflammatory drug (i.e., an NSAID), in oral compositions for preventing and treating periodontal diseases, such as gingivitis. This patent also discloses that steroidal agents, such as hydrocortisone and prednisolone, and certain NSAIDs have reportedly been effective in reducing gingival inflammation when administered orally or topically. However, at least one NSAID, sulindac, is reportedly ineffective in ameliorating gingival inflammation.

U.S. Pat. No. 5,447,725 discloses compositions for aiding periodontal tissue regeneration, which can comprise antimicrobial agents, such as phenolics, and anti-inflammatory agents, such as aspirin, naproxen, ibuprofen, fluribuprofen, indomethacin, eugenol and hydrocortisone. The compositions aid tissue regeneration by hindering infection and inflammation.

U.S. Pat. No. 5,364,616 discloses compositions for prevention and no treatment of gingivitis and periodontitis, comprising antihistamines, which counteract inflammation associated with those conditions. The compositions can further comprise other anti-inflammatory agents, such as NSAIDs, and antimicrobial agents.

Although NSAIDs are, by definition, anti-inflammatory agents, they can actually provoke localized inflammation. Such a side effect is particularly undesirable for oral compositions designed to be topically applied to sensitive mucosal tissues, such as the gingiva. This side effect can be ameliorated by lowering the concentration of NSAIDs applied to the tissue; however, lowering the NSAID concentration also tends to lower the general anti-inflammatory effect of NSAIDs.

The inventors have discovered a synergistic effect in combining an NSAID with the essential oils thymol, methyl salicylate, menthol and eucalyptol. This effect enhances the anti-inflammatory effect of NSAIDs at concentrations insufficient to cause significant localized inflammation, and enhances the anti-microbial effects of eugenol and the essential oils.

All references cited herein are incorporated herein by reference in their entireties.

### SUMMARY OF THE INVENTION

The invention improves upon the prior art by providing an oral composition for treating and/or preventing gingivitis, wherein the composition comprises at least one NSAID, thymol, methyl salicylate, menthol and eucalyptol. The NSAID, thymol, methyl salicylate, menthol and eucalyptol are present in the composition in synergistically effective amounts. The composition can be provided in the form of, e.g., a mouthwash or toothpaste, and is not only effective against gingivitis, but can also prevent or treat halitosis and other detrimental conditions of the oral cavity.

A preferred embodiment of the invention comprises:

about 0.001% to about 2.0 wt. % NSAID;

about 0.02 to about 0.1 wt. % thymol;

about 0.03 to about 0.08 wt. % methyl salicylate;

about 0.03 to about 0.06 wt. % menthol; and

about 0.07 to about 0.11 wt. % eucalyptol.

Preferably, compositions of the invention further comprise:

about 0.1 to about 0.2 wt. % benzoic acid;

about 1.0 to about 55 wt. % of at least one sugar alcohol; and

about 0.01% to about 1.0 wt. %. T127 pluronic, which is a polyoxyethylene-polyoxyproplyene block copolymer (average molecular weight—approx. 12,000), available from BASF Corp.

### DETAILED DESCRIPTION OF PREFERRED EMBODIMENTS

As mentioned above, the inventors have discovered that combining at least one NSAID, thymol, methyl salicylate, menthol and eucalyptol in an oral composition (i.e., a composition adapted for topical application within the oral cavity) renders the oral composition more effective against inflammation and/or plaque inducing microbes than oral compositions containing less than all of the foregoing ingredients. While not intending to be held to any theories, the inventors believe that the synergistic effect results from the combination of ingredients whose similar effects are achieved through different mechanisms.

NSAIDs can be classified according to the following categories (see, e.g., Woodbury et al, “Analgesic-Antipyretic, Antiinflammatory Agents and Drugs Employed in the Treatment of Gout,” in The Pharmacological Basis of Therapeutics, 5th ed., pp. 617-657 (Goodman et al., eds., 1975 pp. 325):

salicylic acid derivatives, such as salicylic acid, acetylsalicylic acid (aspirin), methyl salicylate, diflunisal, salsalate, olsalazine and sulfasalazine;

para-aminophenol derivatives, such as acetaminophen;

indole and indene acetic acids, such as indomethacin, sulindac and etodolac;

fenamates, such as mefenamic, meclofenamic, flufenamic, tolfenamic and etofenamic;

heteroaryl acetic acids, such as tolmetin, ke-orolac and diclofenac;

propionic acid derivatives, such as ibuprofen, naproxen, naproxen sodium, fenoprofen, ketoprofen, flurbiprofen and oxaprozin;

enolic acids, such as the oxicam derivatives piroxicam, meloxicam, ampiroxicam, droxicam, pivoxicam, lornoxicam, cinnoxicam, sudoxicam and tenoxicam, and the pyrazolon derivatives phenylbutazone, oxyphenbutazone, antipyrine, aminopyrine and dipyrone;

alkanones, such as nambumetone;

apazone; and

nimesulide.

NSAIDs suitable for use in the composition of the invention are not particularly limited. In the compositions of the invention, salicylate NSAIDs are preferred and propionic acid derivative NSAIDs are most preferred.

NSAIDs are preferably present in the composition in an amount of about 0.001 to about 2.0 wt. % (unless specified otherwise, all weight percentages herein are with respect to the total weight of the composition), more preferably about 0.01 to about 1.0 wt. %.

Another NSAID that can be used in the composition is eugenol, which is commonly employed as a dental analgesic. Eugenol is preferably present in compositions of the invention in an amount of about 0.001 to about 2.0 wt. %, more preferably about 0.01 to about 1.0 wt. %.

The compositions of this invention preferably include synergistically effective amounts of the essential oils thymol, eucalyptol, menthol and methyl salicylate. Preferably, the total amount of essential oils present in the composition (exclusive of eugenol, which is not considered an essential oil for present purposes although it is sometimes described as such by others) can be from about 0.001 to about 2.0 wt. %, with about 0.01 to about 1.0 wt. % being most preferred.

Thymol is preferably present in the composition in an amount less than about 0.1 wt. %, preferably about 0.02 to about 0.1 wt. %, most preferably about 0.05 to about 0.075 wt. %. Methyl salicylate is preferably present in amounts of about 0.03 to about 0.08 wt. %, most preferably about 0.04 to about 0.07 wt. %. Menthol is preferably present in amounts of about 0.03 to about 0.06 wt. %, most preferably about 0.04 to about 0.05 wt. %. Eucalyptol is preferably present in amounts of about 0.07 to about 0.11 wt. %, most preferably about 0.08 to about 0.10 wt. %.

In addition to these essential oils, benzoic acid is preferably present in amounts of about 0.1 to about 0.2 wt. %, most preferably about 0.13 to about 0.18 wt. %.

Unfortunately, while thymol provides beneficial therapeutic effects, it also provides the consumer with a flavor perception that can be described as unpleasant, harsh or medicinal in taste. Compositions according to the invention preferably mask the harsh taste of thymol, without resorting to additional flavorants, as disclosed in U.S. Pat. No. 4,945,087. The compositions thus preferably incorporate a sugar alcohol, or a mixture of sugar alcohols and anethole.

At least one sugar alcohol usable in the present invention can be any that has effective sweetening capabilities. Preferably, at least one sugar alcohol is selected from the group consisting of sorbitol, xylitol, mannitol, maltitol, hydrogenated starch hydrolysate, and mixtures thereof. More preferably, sorbitol is the sugar alcohol.

At least one sugar alcohol can be present in amounts of about 1.0 to about 55 wt. %, with about 5 to about 50 wt. % being preferred, and about 8 to about 20 wt. % being most preferred.

Anethole is widely used as a flavorant in pharmaceutical compositions. Suitable amounts of anethole in compositions of the invention are usually in the range of about 0.01 to about 0.035 wt. %, with about 0.015 to about 0.025 wt. % being preferred and about 0.018 to about 0.022 wt. % being most preferred.

Suitable liquid forms of the oral compositions of the invention, include, e.g., mouthwashes, sprays and rinses. In such preparations, the vehicle—i.e., the carrier for the ingredients of the mouthwash, such as the essential oils, and the like—is typically a water-alcohol mixture. Generally, the ratio of water to alcohol is in the range of from about 1:1 to about 20:1, preferably about 3:1 to about 20:1 and most preferably about 3:1 to about 10:1 by weight. The total amount of water-alcohol mixture in a mouthwash preparation is typically in the range from about 50 to about 99.9 wt. %.

The pH value of such mouthwash preparations is generally from about 3.5 to about 8.0 and preferably from about 4 to about 7.5. A pH below 3.5 would be irritating to the oral cavity and soften tooth enamel. A pH greater than 8 would result in an unpleasant mouth feel.

Liquid oral preparations may also contain surface active agents—i.e., surfactants—in amounts up to about 5 wt. % and fluorine-providing compounds in amounts up to about 2 wt. %. Surfactants are organic materials that aid in the complete dispersion of the preparation throughout the oral cavity. The organic surface active material may be anionic, non-ionic, ampholytic or cationic.

Suitable anionic surfactants include water-soluble salts of higher fatty acid monoglyceride monosulfates, such as the sodium salt of the monosulfated monoglyceride of hydrogenated coconut oil fatty acids; higher alkyl sulfates, such as sodium lauryl sulfate; alkyl aryl sulfonates, such as sodium dodecyl benzene sulfonate; higher alkyl sulfonacetates; higher fatty acid esters of 1,2-dihydroxy propane sulfonates; and substantially saturated higher aliphatic acyl amides of lower aliphatic amino carboxylic acids such as those having 12 to 16 carbons at the fatty acid, alkyl or acyl radicals. Examples of the last mentioned amides are N-lauroyl sarcosine, and the sodium, potassium, and ethanolamide salts of N-lauroyl, N-myristyl or N-palmitoyl sarcosine.

Suitable non-ionic surfactants include, e.g., poly(oxyethylene)-poly(oxypropylene) block copolymers. Such copolymers are known commercially as poloxamers and are produced in a wide range of structures and molecular weights with varying contents of ethylene oxide and propylene oxide. Suitable non-ionic poloxamers according to the invention are non-toxic and acceptable as direct food additives. They are stable and readily dispersible in aqueous systems and are compatible with a wide variety of formulating ingredients for oral preparations. These surfactants should have an HLB (Hydrophilic-Lipophilic Balance) of between about 10 and 30 and preferably between 10 and 25. Thus, non-ionic surfactants useful in this invention include poloxamers: 105, 108, 123, 124, 183, 184, 185, 188, 215, 217, 234, 235, 237, 238, 284, 288, 334, 335, 338 and 407. These polymers should preferably constitute from 0.05 to 2% by weight of total volume of the liquid oral preparation and preferably from 0.5 to 1% (wt./vol.). A particularly preferred poloxamer is Poloxamer 407 having an HLB of about 22. Such a polymer is sold under the trademark Pluronic F-127 (BASF-WYANDOTTE).

The average molecular weights of the poloxamers listed above are, 105-1,900, 108-5,000, 123-1850, 124-2,200, 183-2,650, 184-2,900, 185-3,400, 188-8,350, 215-4,150, 217-6,600, 234-4,200, 235-4,600, 237-7,700, 238-10,800, 284-4,600, 288-13,500, 334-5,850, 335-6,000, 338-15,000 and 407-12,000.

Another class of non-ionic surfactants useful in this invention are ethoxylated hydrogenated castor oils. Such surfactants are prepared by hydrogenating castor oil and treating the so-formed product with from about 10 to 200 moles of ethylene glycol. They are designated as PEG (numeral) hydrogenated castor oil in accordance with the dictionary of the Cosmetics, Toiletries and Fragrance Association, 3rd Ed., wherein the numeral following PEG indicates the degree of ethoxylation, i.e., the number of moles of ethylene oxide added. Suitable PEG hydrogenated castor oils include PEG 16, 20, 25, 30, 40, 50, 60, 80, 100 and 200. The ethoxylated hydrogenated castor oils are used in the same concentrations as the above described poly(oxyethylene)-poly(oxypropylene) block copolymers.

Other suitable non-ionic surface active agents include condensates of sorbitan esters of fatty acids with from 20 to 60 moles of ethylene oxide (e.g., “Tweens” a trademark of ICI United States, Inc.), and amphoteric agents, such as quaternized imidazole derivatives. Additional acceptable non-ionic surfactants are the condensation products of an alpha-olefin oxide containing 10 to 20 carbon atoms, a polyhydric alcohol containing 2 to 10 carbons and 2 to 6 hydroxyl groups and either ethylene oxide or a heteric mixture of ethylene oxide and propylene oxide. The resultant surfactants are polymers having a molecular weight in the range of 400 to about 1600 and containing 40% to 80% by weight of ethylene oxide, with an alpha-olefin oxide to polyhydric alcohol mole ratio in the range of about 1:1 to 1:3.

Suitable cationic surface active agents are molecules that carry a positive charge, such as cetylpyridinium chloride.

Fluorine providing compounds can be present in the oral preparations of this invention. These compounds may be slightly or fully water soluble and release fluoride ions or fluoride containing ions in water. Typical fluorine providing compounds are inorganic fluoride salts such as soluble alkali metal, alkaline earth metal, and heavy metal salts. For example, sodium fluoride, potassium fluoride, ammonium fluoride, cuprous fluoride, zinc fluoride, stannic fluoride, stannous fluoride, barium fluoride, sodium fluorosilicate, ammonium fluorosilicate, sodium fluorozirconate, sodium monofluorophosphate, aluminum mono- and difluorophosphate and fluorinated sodium calcium pyrophosphate are useful. Alkali metal, tin fluoride and monofluorophosphates such as sodium and stannous fluoride, sodium monofluorophosphate and mixtures thereof are preferred. In an oral liquid preparation, such as a mouthwash, the fluorine providing compound is generally present in an amount sufficient to release up to about 0.15%, preferably about 0.001% to about 0.1% and most preferably from about 0.001% to about 0.05% fluoride by weight of the preparation.

If desired, auxiliary sweeteners can be utilized in the compositions of this invention. Those sweeteners that can be included are those well known in the art, including both natural and artificial sweeteners. Suitable sweeteners include, e.g.:

A. water-soluble sweetening agents such as monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin;

B. water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like;

C. dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerine and L-aspartyl-L-2,5,dihydrophenyl-glycine, L-aspartyl-2,5-dihydro-L-phenylalanine, L-aspartyl-L-(1-cyclohexyen)-alanine, and the like;

D. water-soluble sweeteners derived from naturally occurring water-soluble sweeteners, such as a chlorinated derivative of ordinary sugar (sucrose), known, for example, under the product description of sucralose; and

E. protein based sweeteners such as thaumatococous danielli (Thaumatin I and II).

In general, the effective amount of sweetener used will vary with the sweetener selected and the amount of sweetness desired. This amount will normally be 0.01% to about 40% by weight of the composition when using an easily extractable sweetener. The water-soluble sweeteners described in category A above, are usually used in amounts of about 5 to about 40 wt. %, and preferably in amounts of about 10 to about 20 wt. %. Some of the sweeteners in category A (e.g., glycyrrhizin) can be used in amounts set forth for categories B-E due to the sweeteners' known sweetening ability. In contrast, the sweeteners described in categories B-E are generally used in amounts of about 0.005 to about 5.0 wt. %, with about 0.03 to about 2.5 wt. % being preferred and about 0.03 to about 0.4 wt. % being most preferred. These amounts may be used to achieve a desired level of sweetness independent from the flavor level achieved from any optional flavor oils used.

The use of the sugar alcohols, the essential oils and/or anethole, as discussed above, results in the successful taste masking of the thymol taste. The compositions so masked have a pleasing taste, and, depending on the threshold level of perception of the consumer, may have a pleasing anethole flavor perception. Therefore, additional flavorants or flavors are not necessary; however, if desirable, additional flavorings can be added.

The flavorings that can be used include those known to the skilled artisan, such as, natural and artificial flavors. These flavorings may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, and citrus oil, including lemon, orange, grape, lime and grapefruit and fruit essences including apple, pear, peach, strawberry, raspberry, cherry, plum, pineapple, apricot and so forth.

These flavorings can be used individually or in admixture. Commonly used flavors include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally any flavoring or food additive, such as those described in Chemicals Used in Food Processing, publication 1274 by the National Academy of Sciences, pages 63-258, may be used.

Further examples of aldehyde flavorings include, but are not limited to acetaldehyde (apple); benzaldehyde (cherry, almond); cinnamic aldehyde (cinnamon); citral, i.e., alpha citral (lemon, lime); neral, i.e. beta citral (lemon, lime); decanal (orange, lemon); ethyl vanillin (vanilla, cream); heliotropine, i.e. piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aidehyde (cherry, almond); veratraldehyde (vanilla); 2,6-dimethyl-5-heptenal, i.e. melonal (melon); 2-6-dimethyloctanal (green fruit); and 2-dodecenal (citrus, mandarin); cherry; grape; mixtures thereof; and the like.

The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation. In general, amounts of about 0.05 to about 2.0 wt. % are useable with amounts of about 0.05 to about 1.5 wt. % being preferred.

The compositions of this invention can also contain coloring agents or colorants. The coloring agents are used in amounts effective to produce the desired color. The coloring agents useful in the present invention, include pigments such as titanium dioxide, which may be incorporated in amounts of up to about 2 wt. %, and preferably less than about 1 wt. %. Colorants can also include natural food colors and dyes suitable for food, drug and cosmetic applications. These colorants are known as FD&C dyes and lakes. The materials acceptable for the foregoing spectrum of use are preferably water-soluble, and include FD&C Blue No. 2, which is the disodium salt of 5,5-indigotindisulfonic acid. Similarly, the dye known as Green No. 1 comprises a triphenylmethane dye and is the monosodium salt of 4-[4-N-ethyl-p-sulfobenzyiamino)diphenyl-methylene]-[1-N-ethyl-N-p-sulfonium benzyl)-^{2,5}-cyclo-hexadienimine]. Additional examples include the yellow dye, known as D&C Yellow No. 10, and the dye known as FD&C Green No. 3, which comprises a triphenylmethane dye. A full recitation of all FD&C and D&C dyes and their corresponding chemical structures may be found in the Kirk-Othmer Encyclopedia of Chemical Technology, Volume 5, Pages 857-884, which text is accordingly incorporated herein by reference.

The oral compositions of this invention may also be provided in a substantially solid or pasty form, such as a dental cream, a toothpaste or a toothpowder. Solid or pasty oral preparations contain polishing materials. Typical polishing materials are abrasive particulate materials having particle sizes of up to about 20 microns. Nonlimiting illustrative examples include: water-insoluble sodium metaphosphate, potassium metaphosphate, tricalcium phosphate, dihydrated calcium phosphate, calcium pyrophosphate, magnesium orthophosphate, trimagnesium phosphate, calcium carbonate, alumina, aluminum silicate, zirconium silicates, silica, bentonite, and mixtures thereof.

Polishing materials are generally present in an amount from about 10 to about 82 wt. %. Preferably, they are present in amounts from about 10 to about 75 wt. % in toothpaste, and from about 70 to about 82 wt. % in toothpowder. For toothpaste and dental creams, the water content is about 25 to 50 wt. %. In clear gels, a polishing agent of colloidal silica and alkali metal aluminosilicate complexes is preferred since they have refractive indicies close to the refractive indicies of gelling agent liquid systems commonly used in dentifrices.

In oral preparations that are toothpastes, dental creams, or gels, the liquid vehicle may comprise water, typically in an amount of about 10-90 wt. %. Polyethylene glycol, propylene glycol, glycerin or mixtures thereof may also be present as humectants or binders in amounts of about 20-25 wt. %. Particularly advantageous liquid ingredients comprise mixtures of water with polyethylene glycol or glycerin and propylene glycol. A gelling agent (thickening agent), including natural or synthetic gums such as sodium carboxymethylcellulose, hydroxyethyl cellulose, methyl cellulose and the like may be used, in the range of about 0.5-5% by weight.

In a toothpaste, dental cream or gel, the liquids and solids are proportioned to form a creamy or gelled mass that is extrudable from a pressurized container or from a collapsible tube. The toothpaste or gel may also contain a surface active agent that may be an anionic, nonionic or zwitterionic detergent (surfactant) in amounts of about 0.05-5% by weight. The anionic and nonionic surfactants that are suitable have already been discussed above. Zwitterionic surface active agents include the betaines and sulfobetaines. Typical alkyl dimethyl betaines include decyl betaine or 2-(N-decyl-N,N-dimethylammonio) acetate, coco betaine, myristyl betaine, palmityl betaine, lauryl betaine, cetyl betaine, stearyl betaine, etc. The amidobetaines similarly include cocoamidoethyl betaine, cocoamidopropyl betaine, lauramidopropyl betaine and the like. These sulfobetaines are similar in structure to the betaines, but have a sulfonate group in place of the carboxylate group, and include alkylsulfobetaines, alkylamidosulfobetaines and alkylaminosulfobetaines.

In general, the compositions of this invention are prepared utilizing techniques well known to those skilled in the art. Thus, the liquid compositions may be prepared by mixing the alcohol soluble ingredients with ethanol, adding a quantity of water to the mixture thus obtained, and then blending or mixing in the water soluble ingredients.

For example, in preparing one liter of an embodiment of a liquid oral composition of the invention, eugenol, at least one NSAID, thymol, eucalyptol, menthol, methyl salicylate, anethole, surfactant and benzoic acid are dissolved in and mixed with ethanol. To this resulting mixture a sufficient quantity of water is added, and then the auxiliary sweetener, water soluble colorants, buffers, and the like are blended in. Then additional water is added to make up one liter. Those skilled in the art will appreciate that the total amount of all ingredients used in the compositions of this invention equals 100 wt. % of the total composition.

The invention will be illustrated in more detail with reference to the following Examples, but it should be understood that the present invention is not deemed to be limited thereto.

### EXAMPLE 1

The following indredients can be mixed together to obtain a mouthwash with anti-gingivitis properties. Except as indicated all amounts are in grams.

Thymol 0.639

Sorbitol 300.000

Ethanol 228.000

Eucalyptol 0.922

Menthol 0.425

Benzoic Acid 1.500

Methyl Salicylate 0.552

Poloxamer 407 5.000

Sodium Saccharine 0.300

Sodium Citrate 0.300

Citric Acid 0.100

Color 0.004

Acetaminophen 1.000

Water QS To 1 Liter

### EXAMPLE 2

The following ingredients can be mixed to from a toothpaste.

THYMOL 0.291

METHYL SALICYLATE 0.324

MENTHOL 0.226

EUCALYPTOL 0.389

GLYCERINE 10.000

SORBITOL 36.640

SOLUTION (70%)

WATER 21.339

PEG 400 3.000

XANTHAN GUM 0.900

SODIUM FLUORIDE 0.221

Na SACCHARIN 0.214

SODIUM BENZOATE 0.200

Ti02 0.956

GELLING SILICA 10.000

ABRASIVE SILICA 4.000

SLS 1.300

ACETAMINOPHEN 1.000

While the invention has been described in detail and with reference to specific examples thereof, it will be apparent to one skilled in the art that various changes and modifications can be made therein without departing from the spirit and scope thereof.

What is claimed is:

1. An oral composition comprising at least one NSAID, thymol, methyl salicylate, menthol and eucalyptol.

2. The oral composition of claim 1, wherein said at least one NSAID, thymol, methyl salicylate, menthol and eucalyptol are present in said composition in synergistically effective amounts.

3. The oral composition of claim 1, comprising:
about 0.001 to about 2.0 wt. % of said at least one NSAID;
about 0.02 to about 0.1 wt. % thymol;
about 0.03 to about 0.08 wt. % methyl salicylate;
about 0.03 to about 0.06 wt. % menthol; and
about 0.07 to about 0.11 wt. % eucalyptol.

4. The oral composition of claim 3, further comprising:
about 0.1 to about 0.2 wt. % benzoic acid;
about 20 to about 55 wt. % of at least one sugar alcohol

5. The oral composition of claim 4, wherein said sugar alcohol is selected from the group consisting of sorbitol, xylitol, mannitol, maltitol, hydrogenated starch hydrolsate, and mixtures thereof.

6. The oral composition of claim 4, wherein said sugar alcohol is sorbitol.

7. The oral composition of claim 4, further comprising a surfactant selected from the group consisting of anionic, non-ionic and cationic surfactants.

8. The oral composition of claim 7, wherein said surfactant is a non-ionic surfactant.

9. The oral composition of claim 8, wherein said surfactant is a poloxamer.
